(19) United States
(12) Patent Application Publication
(10) Pub. No.: US 2023/0030895 A1
(43) Pub. Date: Feb. 2, 2023

(54) MAGNETIC RESONANCE IMAGING APPARATUS AND MAGNETIC RESONANCE IMAGING METHOD

(71) Applicant: CANON MEDICAL SYSTEMS CORPORATION, Tochigi (JP)

(72) Inventors: Lijun ZHANG, Beijing (CN); Fanxing MENG, Beijing (CN)

(73) Assignee: CANON MEDICAL SYSTEMS CORPORATION, Tochigi (JP)

(21) Appl. No.: 17/868,960

(22) Filed: Jul. 20, 2022

(30) Foreign Application Priority Data
Jul. 20, 2021 (CN) 202110818036.0
Jun. 15, 2022 (JP) 2022-096815

(51) Int. Cl. A61B 5/055; G01R 33/56

(52) U.S. Cl. CPC A61B 5/055; G01R 33/56; G01R 33/50

(57) ABSTRACT

A magnetic resonance imaging apparatus according to an embodiment includes a processing circuitry configured to generate a pulse sequence including a plurality of repetition times (TRs) each of which includes an echo train and a driven equilibrium pulse applied following the echo train, vary a flip angle of the driven equilibrium pulse, obtain magnetic resonance image data collected by executing the pulse sequence, and reconstruct a magnetic resonance image by using the magnetic resonance image data.

### CROSS-REFERENCE TO RELATED APPLICATIONS

This application is based upon and claims the benefit of priority from Chinese Patent Application No. 202110818036.0, filed on Jul. 20, 2021; and Japanese Patent Application No. 2022-096815, filed on Jun. 15, 2022, the entire contents of all of which are incorporated herein by reference.

### FIELD

Embodiments disclosed herein and in the drawings relate to a magnetic resonance imaging apparatus and a magnetic resonance imaging method.

### BACKGROUND

MRI T1WI-VFA (variable flip angle) 3D is currently the most important pulse sequence for intracranial vessel wall imaging in black blood imaging techniques, and the characteristics of the size, location, structure, contrast, and the like of plaque can be evaluated. In order to better depict the vessel wall, different techniques can also be used to suppress blood and cerebrospinal fluid, and examples of currently commonly used sequences include motion-sensitized driven equilibrium (MSDE), delay alternating with nutation for tailored excitation (DANTE), and inversion recovery-fast spin echo 3D (IR-FSE3D).

MSDE has a motion-sensitive gradient waveform and includes one 90° excitation pulse, a few 180° refocus pulses, and one −90° driven equilibrium pulse, and a gradient field can be applied between pulses to suppress fluid signals.

MSDE intensifies the effect of black blood imaging by suppressing fluid signals such as blood signals, for example, but a signal-to-noise (SN) ratio decreases as an effective TE value (echo delay time) increases.

DANTE is composed of a plurality of flip excitations at low-frequency and can suppress fluid signals. DANTE improves imaging effect of the vessel wall by suppressing fluid signals such as blood and cerebrospinal fluid, for example, but the SN ratio decreases as the flip angle and the number of excitations increase.

IR-FSE3D belongs to an inversion recovery method, and is used to obtain a better contrast of the vessel wall by optimizing the duration from the flip pulse to the next excitation. However, the above described method also reduces the SN ratio of an image. Since high resolution (0.5 mm resolution in each direction) is required in a case of imaging some specific areas, for example, intracranial vessel wall imaging and the like, it is also more difficult to obtain a good SN ratio.

### BRIEF DESCRIPTION OF THE DRAWINGS

FIG. 1 is a diagram illustrating a magnetic resonance imaging apparatus according to a first embodiment;

FIG. 2 is a diagram illustrating a pulse sequence generated by a sequence generation unit in the magnetic resonance imaging apparatus according to the first embodiment;

FIG. 3 is a diagram illustrating signal calibration processing in the magnetic resonance imaging apparatus according to the first embodiment;

FIG. 4 is a flowchart illustrating the procedure of a magnetic resonance imaging method for the magnetic resonance imaging apparatus according to the first embodiment;

FIGS. 5A, 5B, and 5C are effect contrast diagrams in a case of performing magnetic resonance imaging by using pulse sequences different from each other;

FIG. 6 is a diagram illustrating a magnetic resonance imaging apparatus according to a second embodiment;

FIG. 7 is a diagram illustrating signal calibration processing in the magnetic resonance imaging apparatus according to the second embodiment; and

FIG. 8 is a flowchart illustrating the procedure of a magnetic resonance imaging method for the magnetic resonance imaging apparatus according to the second embodiment.

### DETAILED DESCRIPTION

A magnetic resonance imaging apparatus according to an aspect of the present embodiment includes processing circuitry that is configured to generate a pulse sequence including repetition times (TRs) each of which includes an echo train and a driven equilibrium pulse applied following the echo train, vary a flip angle of the driven equilibrium pulse, obtain magnetic resonance image data collected by executing the pulse sequence, and reconstruct a magnetic resonance image by using the magnetic resonance image data.

Hereinbelow, details of embodiments of the magnetic resonance imaging apparatus and a magnetic resonance imaging method will be described with reference to the drawings.

Hereinbelow, the details of preferred embodiments of the magnetic resonance imaging apparatus and the magnetic resonance imaging method of the present disclosure will be described with reference to the drawings. Herein, the same numerals and signs are given to identical or similar configurations, and as appropriate, duplicate explanations will not be repeated.

### First Embodiment

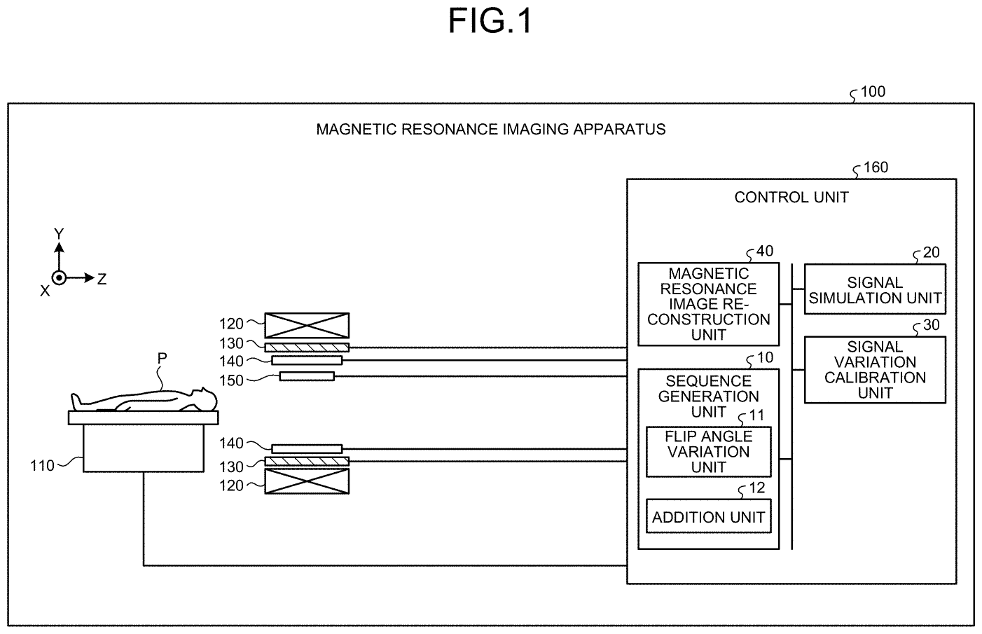

FIG. 1 is a structural block diagram of a magnetic resonance imaging apparatus according to a first embodiment.

As illustrated in FIG. 1, a magnetic resonance imaging apparatus 100 is mainly provided with a couch 110, a static magnetic field magnet 120, a gradient coil 130, a transmitter coil 140, a receiver coil 150, and a control unit 160.

The couch 110 is used for a subject P to be placed in a collection area of a magnetic resonance apparatus. The static magnetic field magnet 120 is used to generate a static magnetic field in a space of the magnetic resonance imaging apparatus 100 for detecting a subject.

The gradient coil 130 is disposed inside the static magnetic field magnet 120. The gradient coil 130 is formed by combining three coils corresponding to X, Y, and Z axes, respectively, which are orthogonal to each other, and these three coils can individually generate gradient magnetic fields in which intensities of the magnetic fields vary along the X, Y, and Z axes under the control of the control unit 160. The gradient magnetic fields in the X, Y, and Z axes generated by the gradient coil 130 are, for example, a readout gradient field Gx, phase-encoding gradient field Gy, and slice gradient field Gz, respectively.

The transmitter coil 140 is disposed inside the gradient coil 130 and generates a radio frequency magnetic field. The control unit 160 supplies RF pulses corresponding to Larmor frequencies determined depending on the type of atom under investigation and the intensity of the magnetic field to the transmitter coil 140.

The receiver coil 150 is disposed inside the gradient coil 130 and receives a magnetic resonance signal (hereinafter, may also be referred to as an echo signal or a collection signal) emitted from the subject P under the influence of the radio frequency magnetic field. In a case of receiving the magnetic resonance signal, the receiver coil 150 outputs the received magnetic resonance signal to the control unit 160.

The control unit 160 controls individual parts of the magnetic resonance imaging apparatus 100, which include a processor, a memory, and various circuits such as a transmitting circuit and a receiving circuit.

The contents mainly involved in the present embodiment are a configuration and a method of processing a pulse sequence used in the magnetic resonance imaging apparatus and a magnetic resonance image data collected, which is achieved by a processor in the magnetic resonance imaging apparatus executing each functional module stored in the memory. Therefore, in the control unit 160 in FIG. 1, only each functional module according to the first embodiment of the present embodiment is illustrated, and assuming that the control unit 160 is composed of individual functional modules, other components are omitted. The magnetic resonance imaging apparatus may also include other hardware structures and other control modules in the control unit 160, but these are not described herein since all of which can be achieved by configurations in the related art.

Each functional module described in the present specification corresponds to processing executed by a processor (processing circuitry), and may be implemented as software installed in the memory of the magnetic resonance imaging apparatus and executed by the processor reading and executing a software program in the memory. Each functional module may also be implemented as hardware to be formed in a dedicated circuit with a corresponding function and incorporated into the magnetic resonance imaging apparatus. The magnetic resonance imaging apparatus can receive, transmit, and collect data via networks such as the Internet.

The term “processor” as used in the above description means, for example, a circuit such as central processing unit (CPU), graphical processing unit (GPU), application specific integrated circuit (ASIC), PLDs (for example, Simple Programmable Logic Device (SPLD) and Complex Programmable Logic Device (CPLD)), or Field Programmable Gate Array (FPGA).

Instead of storing the software program in the memory, the software program may be configured to be incorporated directly into a circuit of the processor. In this case, the processor implements its function by executing the program incorporated in the circuit.

Returning to the explanation in FIG. 1. As illustrated in FIG. 1, functional modules of the control unit 160 include sequence generation unit 10, a signal simulation unit 20, a signal variation calibration unit 30, and a magnetic resonance image reconstruction unit 40.

The sequence generation unit 10, a flip angle variation unit 11, an addition unit 12, the signal simulation unit 20, the signal variation calibration unit 30, and the magnetic resonance image reconstruction unit 40 illustrated in FIG. 1 are implemented by the processor (processing circuitry) described above as a sequence generation function, a flip angle variation function, an addition function, a signal simulation function, a signal variation calibration function, and the magnetic resonance image reconstruction unit 40, respectively. The sequence generation unit 10, the flip angle variation unit 11, the addition unit 12, the signal simulation unit 20, the signal variation calibration unit 30, and the magnetic resonance image reconstruction unit 40 are examples of a generation unit, a variation unit, an addition unit, a simulation unit, a calibration unit, and a reconstruction unit, respectively.

Among these, the sequence generation unit 10 generates a pulse sequence for collecting magnetic resonance image data. The pulse sequence is a pulse process and a combination of pulses used by the magnetic resonance apparatus during an examination, and in the first embodiment, the sequence generated by the sequence generation unit 10 is required to satisfy predetermined conditions as follows: the pulse sequence includes a plurality of repetition times TR; each repetition time includes an echo train and a driven equilibrium pulse that follows the echo train. Since T1WI, which is a longitudinal relaxation time T1-weighted sequence that is currently commonly used for intracranial vessel wall imaging and the like, satisfies the conditions described above, the T1WI sequence is adopted as a base sequence, and collecting intracranial vessel wall images is employed as an example.

The sequence generation unit 10 can generate a normal T1WI sequence, and in a T1WI sequence in the related art, the tail of each of the repetition times TR includes the same driven equilibrium pulse. In other words, the sequence generation unit 10 executes the longitudinal relaxation time T1-weighted sequence (T1WI sequence) through the sequence generation function implemented by the processing circuitry. The sequence generation unit 10 generates a pulse sequence including repetition times TR each of which includes an echo train and a driven equilibrium pulse applied following the echo train through the sequence generation function implemented by the processing circuitry. The sequence generation unit 10 is further provided with the flip angle variation unit 11 that can correct the pulse sequence and the addition unit 12. In other words, the sequence generation unit 10 executes the pulse sequence including the repetition times TR each of which includes an echo train and a driven equilibrium pulse applied following the echo train, while varying the flip angle of the driven equilibrium pulse.

Specifically, the flip angle variation unit 11 varies a flip angle of a driven equilibrium pulse at the tail of each of the repetition times TR in the T1WI sequence to generate a difference between flip angles of driven equilibrium pulses present at different repetition times TR. In other words, the flip angle variation unit 11 varies the flip angles of the driven equilibrium pulses included in the longitudinal relaxation time T1-weighted sequence (T1WI sequence) through the flip angle variation function by the processing circuitry.

For example, the flip angle variation unit 11 sets up the flip angle of the driven equilibrium pulse at each of the repetition times TR so that an angle decreases as each of the repetition times TR elapses. Alternatively, the flip angle variation unit 11 may set up the flip angle of the driven equilibrium pulse at each of the repetition times TR through the flip angle variation function implemented by the processing circuitry so that the flip angle of the driven equilibrium pulse decreases exponentially as each of the repetition times TR elapses. The flip angles of the driven equilibrium pulses can also be varied randomly by the flip angle variation unit 11, rather than following a predetermined rule. In order to optimize a signal-to-noise (SN) ratio, the flip angle variation unit 11 preferably gradually decreases the flip angle of the driven equilibrium pulse with the lapse of time through the flip angle variation function implemented by the processing circuitry.

The addition unit 12 adds a single inversion recovery (IR) pulse TI at the beginning of the entire T1WI sequence by having a time.

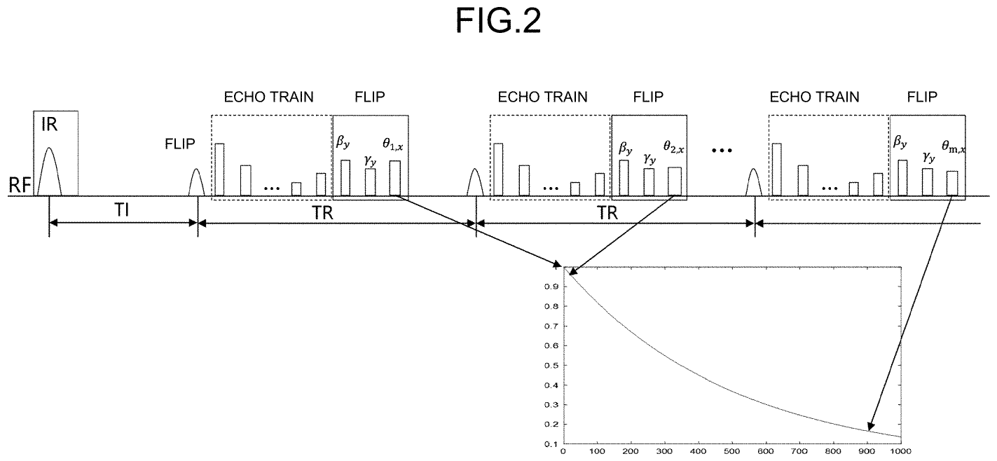

FIG. 2 is a schematic diagram illustrating a pulse sequence (RF) finally generated by the sequence generation unit 10 through variation performed by the flip angle variation unit 11 and an addition unit 12.

First, a schematic time-series diagram representing the pulse sequence is illustrated at the upper side of FIG. 2. As illustrated in FIG. 2, a single IR pulse is first provided at the beginning of the pulse sequence, and followed by the single IR pulse, after the time TI, a plurality of the repetition times TR are concatenated. Thereby, when viewed on the time axis, the pulse sequence consists of the TI and the TRs. Here, the TI is about 2 seconds, but TI is not limited to 2 seconds and can be any other length of time period. Each TR is usually less than 1 second, but in a case where the TI is set to about 2 seconds, an excitation signal level increases more in the case of TI than in the case of TR.

Each of the repetition times TR starts with excitation by one flip pulse and mainly includes an echo train and a set of flip angles. The echo train in FIG. 2 is surrounded by a dashed line frame and includes a plurality of refocus pulses, and the composition of the echo train is identical to the T1WI sequence. Put it in simple terms, each of columnar bars within the dashed frame in FIG. 2 represents a single refocus pulse, and different heights of the columnar bars correspond to different pulse intensities (also referred to as pulse angles). Since the magnetic resonance imaging apparatus usually collects an echo signal following each refocus pulse, the number of refocus pulses is equal to the number of signal collections, and a combination of echoes following the refocus pulses are referred to as an echo train.

The set of the flip angles includes flip angles β_{y}, γ_{y}, and θₓ, and the area surrounded by the solid line frame after the echo train in FIG. 2 illustrates the set of the flip angles, where three columnar bars represent flip angles β_{y}, γ_{y}, and θₓ, respectively, and different heights of the columnar bars correspond to different angles. Among these, the flip angles β_{y} and γ_{y} are flip angles that transfer the magnetization vector remaining after the echo train is applied to the transverse plane. The flip angle θₓ is a flip angle that is provided at the tail of each repetition time TR and allows the remaining transverse magnetization vector to be flipped to a negative Z axis, thereby improving T1 contrast, which is referred to as a flip angle of the driven equilibrium pulse. Here, the flip angles θₓ in the repetition times TR are denoted by θ_{1,x} to θ_{m,x}, respectively (where m is a natural number equal to or greater than 1 and represents what number repetition time TR). Thereby, a flip angle θₓ at a first repetition time TR following the TI is, for example, θ_{1,x}.

In a case where the flip angles β_{y}, and γ_{y} are provided, the effect of the flip angle of the driven equilibrium pulse can be enhanced. However, only the flip angle of the driven equilibrium pulse may be provided without the flip angles β_{y}, and γ_{y}. The flip angle variation unit 11 is configured to exponentially decrease the flip angles θ_{1,x} to θ_{m,x} of driven equilibrium pulses as illustrated in the chart at the lower side of FIG. 2. That is, the flip angles θ_{1,x} to θ_{m,x} of the driven equilibrium pulses are the points on the exponentially decreasing curve in the chart in FIG. 2, as indicated by the arrows. The horizontal coordinate in the chart represents m, and the vertical coordinate represents signal intensities. From the time series diagram of the pulse sequence in FIG. 2, it can be intuitively seen that the heights of the columnar bars of the flip angles θ_{m,x} of the driven equilibrium pulses in the repetition times TR decrease as those are toward the right.

The exponential equation for adjusting the flip angles is, for example, as in Equation (1) below.

$${α}_{n} = {α}_{0} {e}^{- \frac{n}{N}} \qquad ( 1 )$$

In Equation (1), α₀ represents an initial (n=0) flip angle and is usually set to 90°, and the parameter n is a natural number equal to or greater than 0, which is from 0 to the number of flip angles of the driven equilibrium pulses. The parameter N is a parameter for controlling a decreasing rate and is generally set to half of the total amount of the repetition times TR in order to maintain equilibrium between the SN ratio and contrast.

Therefore, the sequence generation unit 10 generates and outputs a pulse sequence in which the flip angles of the driven equilibrium pulses at the tails of the repetition times TR vary exponentially, as illustrated in FIG. 2. The control unit 160 controls a configuration of the couch 110, the gradient coil 130, the transmitter coil 140, the receiver coil 150, and the like according to the pulse sequence finally generated by the sequence generation unit 10, collects echo signals from the subject P, and transmits the collected echo signals to the magnetic resonance image reconstruction unit 40 as raw data of a magnetic resonance image. These collected echo signals may be referred to as collected signals.

In the first embodiment, the flip angles of the driven equilibrium pulses are provided to vary exponentially, thereby capable of obtaining the higher SN ratio than those in the related art. On the other hand, point diffusion or artifacts may occur due to signal variations. Thus, a magnetic resonance image can also be further optimized by calibrating the collected signals.

Specifically, the signal simulation unit 20 simulates an excitation signal in each of the repetition times TR using the signal simulation function implemented by the processing circuitry to obtain a simulation signal. For example, the signal simulation unit 20 can simulate an amplitude of a flip signal at the beginning of each repetition time TR using the Bloch equation or the extended phase graph (EPG) method through the signal simulation function implemented by the processing circuitry to calculate the amplitude of the simulation signal. For example, the Bloch equation is a set of macroscopic equations, and in a case where the pulse sequence is known, the simulation signal can be evaluated by the Bloch equation. For example, in a case where relaxation times T1 and T2 are given, the Bloch equation is used to calculate a nuclear magnetization intensity M=(Mx, My, Mz) as a function of time, thereby capable of representing vector variations under conditions of a certain RF field, a gradient field, a relaxation time, and the like. The extended phase graph (EPG) algorithm is also commonly used for signal simulation. As a matter of course, the signal simulation algorithm for a pulse sequence is not limited to the two types described above; it is possible to simulate the pulse sequence by using each of the existing simulation methods.

The signal variation calibration unit 30 calibrates the magnetic resonance image data obtained by the magnetic resonance image reconstruction unit 40 using the simulation signal simulated by the signal simulation unit 20 through the signal variation calibration function implemented by the processing circuitry. Specifically, the signal variation calibration unit 30 calculates, from the simulation signal simulated by the signal simulation unit 20, a reference value for this simulation signal, defines a ratio of the simulation signal to this reference value as a weighting value, and assigns the weighting value thus obtained to the collected signals obtained by the magnetic resonance imaging apparatus executing this sequence, thereby calibrating the collected signals and transmitting the calibrated collected signals to the magnetic resonance image reconstruction unit 40.

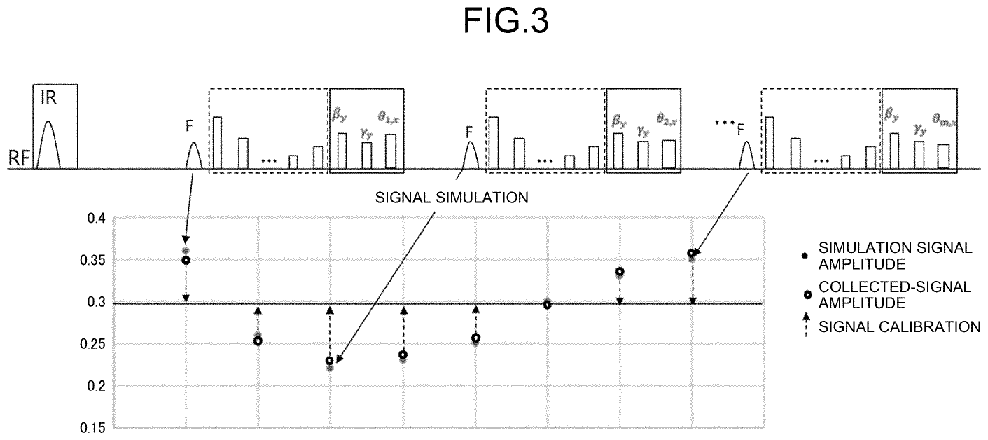

FIG. 3 is a schematic diagram illustrating signal calibration processing in the magnetic resonance imaging apparatus according to the first embodiment. Here, the upper side of FIG. 3 illustrates a pulse sequence generated by the sequence generation unit 10, and the lower side of FIG. 3 illustrates a schematic chart of the calibration processing executed by the signal variation calibration unit 30. In the chart, solid circles represent amplitudes of simulation signals obtained by simulating flip pulses F at the beginning of the repetition times TR connected to the solid arrows, and correspond to the intensities of the flip pulses F. Based on the simulation results, simulation results of the individual flip pulses F are mapped onto the chart illustrated in FIG. 3 in which the horizontal axis is time and the vertical axis is amplitude to form a plurality of solid circles. Based on the amplitude of each simulation signal that has been simulated, an average line at position 0.3 is obtained by an average value of the individual amplitudes, that is, the average value of the simulation signals is 0.3, and this average value is divided by the amplitude of each simulation signal, to obtain a calibration ratio r. Hollow circles of icons represent amplitudes of the collected signals actually collected, and each of the collected signals is calibrated by multiplying the calibration ratio r by each of the amplitudes of the collected signals to bring each of the collected signals closer to the mean line, as illustrated by the dashed lines in the figure. The calibration processing in FIG. 3 is schematic; in practice, since an echo train in a typical pulse sequence usually contains a plurality of the refocus pulses and collects an echo following each refocus pulse, a plurality of the collected signals are provided at each of the repetition times TR, and during calibration, all of the collected signals are multiplied by the calibration ratio r of each of the repetition times TR, thereby performing calibration.

Where the calibration ratio is r, the simulation signal amplitude is Ss, and the collected-signal amplitude is Sa, as described above, equations of a calibration relationship are as follows.

Calibration ratio(r)=Average value/Ss

Signal value that has been calibrated=Sa*r=Sa*(average value/Ss)

Here, although the average value is explained as the reference value for the simulation signal, the reference value is not limited to the average value, and another calculation method that adopts an intermediate value or the like may be used.

The magnetic resonance image reconstruction unit 40 can obtain magnetic resonance image data collected by executing a pulse sequence generated by the sequence generation unit 10 and reconstruct a magnetic resonance image by using the magnetic resonance image data. That is, the magnetic resonance image reconstruction unit 40 obtains magnetic resonance image data collected by executing a pulse sequence through the magnetic resonance image reconstruction function implemented by the processing circuitry and reconstructs a magnetic resonance image by using the magnetic resonance image data.

In a case where collected signals that have been calibrated exists, the magnetic resonance image reconstruction unit 40 reconstructs the magnetic resonance image by using the collected signals that have been calibrated. For example, the magnetic resonance image reconstruction unit 40 reconstructs an image by filling k-space with echo signals collected by the magnetic resonance imaging apparatus. Known magnetic resonance image reconstruction methods may be used for image reconstruction, and the details are not described.

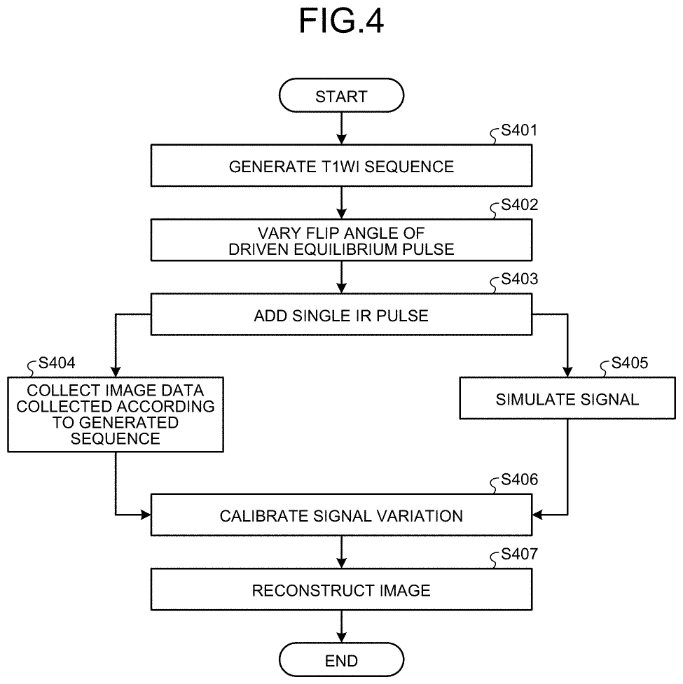

FIG. 4 is a flowchart illustrating a magnetic resonance imaging method for the magnetic resonance imaging apparatus according to the first embodiment.

First, the sequence generation unit 10 generates the T1WI sequence by using a known sequence generation method (step S401), where the sequence generation unit 10 may generate the T1WI sequence by directly obtaining the T1WI sequence that satisfies requirements via external facilities or networks.

Next, at a step S402, the flip angle variation unit 11 varies the flip angle of the driven equilibrium pulse provided at the tail of each of the repetition times TR in the T1WI sequence that has been obtained at the step S401 so as to exponentially decrease the flip angle with the lapse of the repetition times TR. The addition unit 12 adds and installs the single IR pulse at the beginning of the entire T1WI sequence by having the time TI (step S403). That is, the sequence generation unit 10 adds the single IR pulse at the beginning of the pulse sequence composed of the repetition times TR through the sequence generation function by the processing circuitry to generate the pulse sequence to be executed.

Next, at a step S404, the magnetic resonance imaging apparatus 100 collects echo signals according to sequence pulses finally generated by the sequence generation unit 10 through variation by the flip angle variation unit 11 and the addition unit 12, and the collected signals are used as magnetic resonance image data.

Furthermore, at a step S405, the signal simulation unit 20 simulates excitation signals in the repetition times TR by using the Bloch equation to obtain simulation signals.

Next, the signal variation calibration unit 30 obtains the collected signals collected at the step S404 and the simulation signals calculated at the step S405, calculates a calibration ratio by using the simulation signals, and assigns the calibration ratio as a weighting value to the collected signals to obtain collected signals that have been calibrated (step S406).

Next, proceeding to the step S407, the magnetic resonance image reconstruction unit 40 reconstructs a magnetic resonance image based on the collected signals that have been calibrated.

The order of execution of the steps S402 and S403 in the flowchart in FIG. 4 is not limited to this order, and the flip angle of the driven equilibrium pulse may be varied after adding the single IR pulse.

Furthermore, by combining the steps S401, S402, and S403 to be one step, the sequence generation unit 10 can directly generate a pulse sequence in which flip angles of driven equilibrium pulses are varied according to rules for generating the T1WI sequence and for varying flip angles of driven equilibrium pulses.

According to the first embodiment, by the flip angles of the driven equilibrium pulses in the pulse sequence varying with the lapse of time, the SN ratio of the image can be increased, image contrast of the image can be maintained, and the quality of reconstruction of the image can be improved. The SN ratio of the image can be further increased by adding the single IR pulse at the beginning of the pulse sequence. Furthermore, image defects such as artifacts due to signal variation can be reduced by the simulation of univariate and the calibration of the collected signals using the simulation signals.

### Modified Example of First Embodiment

In the first embodiment, although the flip angles of the driven equilibrium pulses in the pulse sequence are allowed to gradually vary with the lapse of time, and the single IR pulse is added at the beginning of the sequence, only the flip angles of the driven equilibrium pulses may be allowed to vary, and addition of the single IR pulse may be omitted. In a case where only the flip angles of the driven equilibrium pulses in the pulse sequence are allowed to sequentially vary with the lapse of time, the SN ratio of the image can be increased, and contrast of the image can be maintained as compared to the related art.

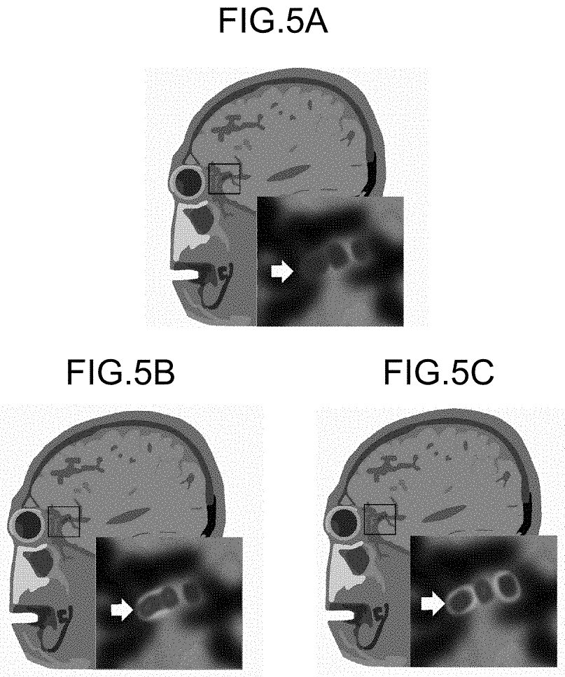

FIGS. 5A, 5B, and 5C are effect contrast diagrams in a case of performing magnetic resonance imaging by using pulse sequences different from each other. FIG. 5A is a diagram illustrating an image reconstructed based on signals collected from the T1WI sequence in the related art, and the depiction on a vessel wall is not clear as indicated by a blank arrow since the SN ratio is low.

FIG. 5B is a diagram illustrating an image collected and reconstructed after varying only the flip angles of the driven equilibrium pulses in the T1WI sequence so as to exponentially decrease the flip angles, and in this image, a SN ratio of the vessel wall is improved as compared to the SN ratio of the image illustrated in FIG. 5A, and the vessel wall is clearly visible. FIG. 5C is a diagram illustrating an image collected and reconstructed after varying the flip angles of the driven equilibrium pulses in the T1WI sequence so as to exponentially decrease the flip angles and adding the single IR pulse at the beginning of the sequence. In this image, a SN ratio of the vessel wall is significantly improved as compared to that of the image illustrated in FIG. 5A, and the vessel wall is more clearly visible. From the above comparison, the different effects presented by the different variation schemes can be intuitively seen. As described above, even in the case where only the flip angles of the driven equilibrium pulses are varied, the similarly significant improvement can be obtained.

In the first embodiment, the explanation is based on the T1WI in the related art, but the present embodiment is not limited thereto, and the present embodiment may be applied to other pulse sequences as long as the sequence includes an echo train and a driven equilibrium pulse. In addition, in the first implementation method, the signal simulation unit 20 and the signal variation calibration unit 30 are used to calibrate the collected signals to eliminate image defects such as artifacts, but the signal simulation unit 20 and the signal variation calibration unit 30 may not be employed, and the pulse sequence that has been corrected is directly used to collect signals. The collected signals still have certain technical effects as compared to signals collected by methods in the related art.

### Second Embodiment

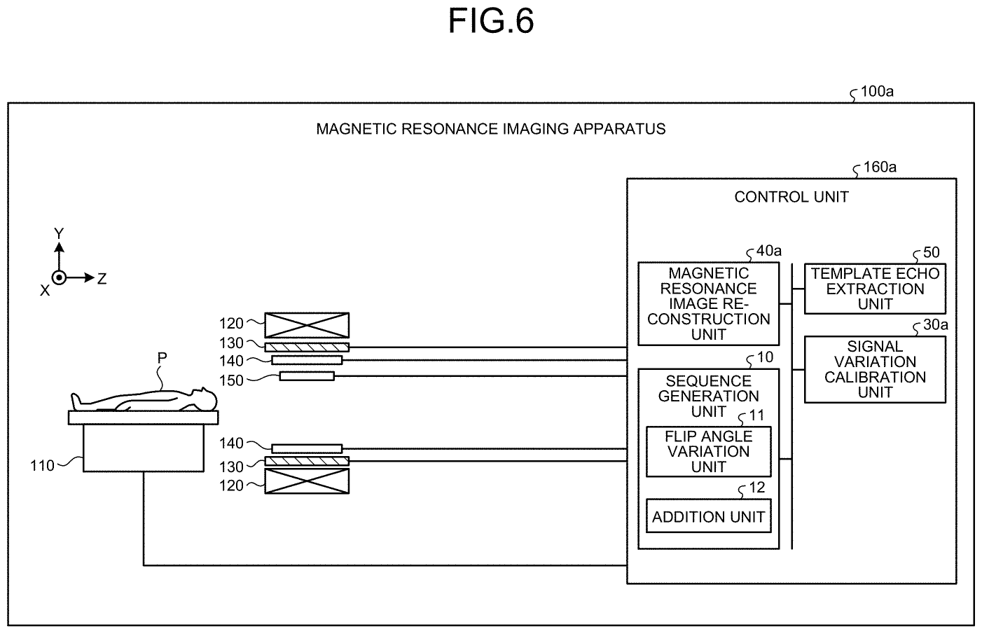

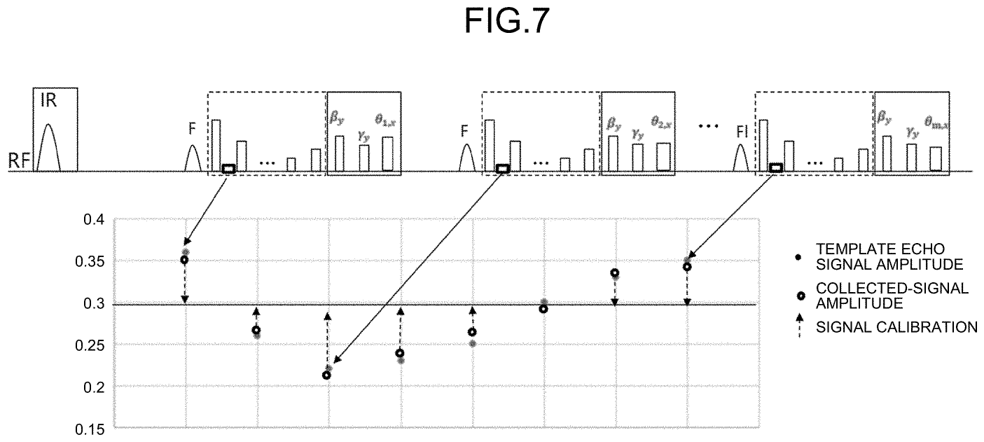

A second embodiment is explained with reference to FIGS. 6 and 7. In a case of comparing a magnetic resonance imaging apparatus according to the second embodiment with that of the first embodiment, the difference lies in a different method of signal calibration. Hereinbelow, the difference will be explained, and duplicate explanations will not be repeated.

FIG. 6 is a structural block diagram of a magnetic resonance imaging apparatus according to a second embodiment. As illustrated in FIG. 6, a magnetic resonance imaging apparatus 100a is mainly provided with the couch 110, the static magnetic field magnet 120, the gradient coil 130, the transmitter coil 140, the receiver coil 150, and a control unit 160a. As functional modules included in the control unit 160, the sequence generation unit 10, a magnetic resonance image reconstruction unit 40a, a template echo extraction unit 50, and a signal variation calibration unit 30a are provided.

Similar to FIG. 1, the sequence generation unit 10, the flip angle variation unit 11, the addition unit 12, the signal variation calibration unit 30a, the magnetic resonance image reconstruction unit 40a, and the template echo extraction unit, which are illustrated in FIG. 6, are implemented by the processor (processing circuitry) as the sequence generation function, the flip angle variation function, the addition function, the signal variation calibration function, a magnetic resonance image reconstruction function, and a template echo extraction function, respectively. The sequence generation unit 10, the flip angle variation unit 11, the addition unit 12, the signal simulation unit 20, the signal variation calibration unit 30a, the magnetic resonance image reconstruction unit 40a, and the template echo extraction unit 50 are examples of a generation unit, a variation unit, an addition unit, a simulation unit, a calibration unit, a reconstruction unit, and an extraction unit, respectively.

The sequence generation unit 10 generates the T1WI sequence and further includes the flip angle variation unit 11 and the addition unit 12, causes the flip angle variation unit 11 to vary a flip angle of a drive equilibrium sequence provided at the tail of each of the repetition times TR in the T1WI sequence, and causes the addition unit 12 to add a single IR pulse at the beginning of the entire T1WI sequence by having the time TI.

The magnetic resonance image reconstruction unit 40a can obtain magnetic resonance image data collected by executing a pulse sequence generated by the sequence generation unit 10 and reconstruct a magnetic resonance image by using the magnetic resonance image data. In a case where collected signals that have been calibrated exists, the magnetic resonance image reconstruction unit 40a reconstructs the magnetic resonance image by using the collected signals that have been calibrated. Accordingly, a detailed explanation is not repeated herein.

In the second embodiment, the magnetic resonance imaging apparatus 100a calibrates the collected signals by using the template echo extraction unit 50 and the signal variation calibration unit 30a, thereby performing calibration. An echo train of the pulse sequence has the refocus pulses, and one echo collection is performed following each refocus pulse. The template echo extraction unit 50 selects a signal that has been collected by echo collection as a template echo from the echo collection corresponding to each refocus pulse. For example, the template echo extraction unit 50 selects an echo signal that has been collected by the echo collection corresponding to a first refocus pulse of the echo train, as a template echo.

In a case where the template echo is selected from the collected echo signals, the control unit 160a controls the gradient coil 130 not to phase encode with respect to the template echo during the execution of the pulse sequence by the gradient coil 130, and furthermore, does not use the template echo during the image reconstruction.

The template echo extraction unit 50 selects an echo signal collected in the echo collection corresponding to the first refocus pulse of the echo train as the template echo and uses this template echo for signal calibration only, while the magnetic resonance image reconstruction unit 40a selects an echo signal collected in the echo collection corresponding to a subsequent refocus pulse following a second refocus pulse as raw data of the magnetic resonance image and uses the raw data for image reconstruction.

The signal variation calibration unit 30a calibrates magnetic resonance image data other than the template echoes obtained by the magnetic resonance image reconstruction unit 40 using the template echoes extracted by the template echo extraction unit 50. Specifically, the signal variation calibration unit 30a calculates, from the template echoes extracted by the template echo extraction unit 50, a reference value for this template echo signal, defines a ratio of the template echo signal to this reference value as a weighting value, and assigns the weighting value thus obtained to the raw data of the magnetic resonance imaging apparatus, thereby performing calibration.

FIG. 7 is a schematic diagram illustrating signal calibration processing in the magnetic resonance imaging apparatus of the second embodiment. Here, the upper side of FIG. 7 illustrates a pulse sequence generated by the sequence generation unit 10, and in the pulse sequence, each of blocks following a columnar bar indicating the first refocus pulse of the echo train represents an echo collection performed following the first refocus pulse, where an echo signal collected in this echo collection is the template echo. A schematic chart of the calibration processing executed by the signal variation calibration unit 30a is illustrated at the lower side of FIG. 7. In the chart, solid circles represent amplitudes of the template echoes connected to the solid arrows, and correspond to the intensities of the template echoes. In a case where each template echo is mapped onto the chart as illustrated in FIG. 7 in which the horizontal axis is time and the vertical axis is amplitude, a plurality of the solid circles are formed. Based on the amplitude of the template echo in each of the repetition times TR, an average value of the individual amplitudes is obtained. For example, an average line is formed at position 0.3, that is, the average value of the amplitudes of the template echoes is 0.3, and this average value is divided by each of the amplitudes of each of the template echoes, to obtain a calibration ratio r. Hollow circles of icons represent amplitudes of other collected signals other than the template echoes actually collected, and each of the collected signals is calibrated by multiplying the calibration ratio r by each of the amplitudes of the collected signals to bring each of the collected signals closer to the mean line, as illustrated by the dashed lines in the figure. The calibration processing in FIG. 7 is schematic; in practice, since an echo train in a typical pulse sequence usually contains a plurality of the refocus pulses and collects an echo following each refocus pulse, a plurality of the collected signals are provided at each of the repetition times TR, and during calibration, all of the collected signals other than the template echoes are multiplied by the calibration ratio r of each of the repetition times TR, thereby performing calibration.

Where the calibration ratio is r, the template echo amplitude is St, and the collected-signal amplitude is Sa, as described above, equations of a calibration relationship are as follows.

Calibration ratio(r)=Average value/St

Signal value that has been calibrated=Sa*r=Sa*(average value/St)

Here, although the average value is explained as the reference value for the template echo, the reference value is not limited to the average value, and another calculation method that adopts an intermediate value or the like may be used.

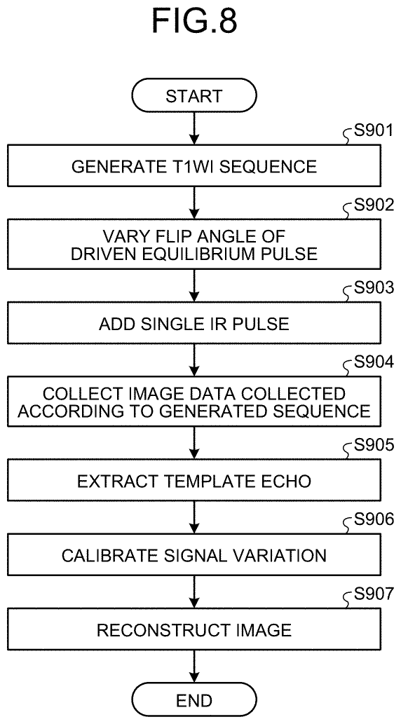

FIG. 8 is a flowchart illustrating a magnetic resonance imaging method for the magnetic resonance imaging apparatus according to the second embodiment. First, the sequence generation unit 10 generates the T1WI sequence by using a known sequence generation method (step S901), where the sequence generation unit 10 can generate the T1WI sequence by directly obtaining the T1WI sequence that satisfies requirements via external facilities or networks.

Next, at a step S902, the flip angle variation unit 11 varies the flip angle of the driven equilibrium pulse provided at the tail of each of the repetition times in the T1WI sequence that has been obtained at the step S901 so as to exponentially decrease the flip angles with the lapse of the repetition times TR. The addition unit 12 adds and installs the single IR pulse by having the time T1 at the beginning of the entire T1WI sequence (step S903).

Next, at a step S904, the magnetic resonance imaging apparatus 100a collects echo signals according to sequence pulses finally generated by the sequence generation unit 10 through variation by the flip angle variation unit 11 and the addition unit 12, thereby the magnetic resonance image reconstruction unit 40a collecting magnetic resonance image data that have been collected according to the generated sequence as the collected signals.

Next, at a step S905, the template echo extraction unit 50 extracts, from the echo signals collected at the step S904, an echo collected following the first refocus pulse in each of the repetition times TR as a template echo, thereby the signal variation calibration unit 30a calculating a calibration ratio by using the template echo signal and assigning this calibration ratio to another collected signal other than the template echo as a weighting value to perform calibration, and the collected signal that has been calibrated is obtained (step S906). That is, the template echo extraction unit 50 extracts one echo from the echo train in each of the repetition times TR as the template echo with the template echo extraction function implemented by the processing circuitry. The signal variation calibration unit 30a calibrates the magnetic resonance image data obtained by the magnetic resonance image reconstruction unit 40a using the template echo through the signal variation calibration function implemented by the processing circuitry.

Next, proceeding to the step S907, the magnetic resonance image reconstruction unit 40a reconstructs a magnetic resonance image based on the collected signals that have been calibrated.

The order of execution of the steps S902 and S903 in the flowchart in FIG. 8 is not limited to this order, and the flip angle of the driven equilibrium pulse may be varied after adding the single IR pulse.

Furthermore, by combining the steps S901, S902, and S903 to be one step, the sequence generation unit 10 may directly generate a pulse sequence in which flip angles of driven equilibrium pulses are varied according to rules for generating the T1WI sequence and for varying the flip angles of the driven equilibrium pulses.

According to the second embodiment, by directly using any collected echo signals to calibrate the other echo signals, a large amount of computation during echo simulation can be avoided, computing resources can be saved, and image defects such as artifacts due to signal variations can be reduced by calibration of the collected signals.

Each functional module in the magnetic resonance imaging apparatus described in the above embodiments can be executed as a software program. By storing and reading this program in advance, a general-purpose computer can achieve the same effect as that achieved by the magnetic resonance imaging apparatuses of the above described embodiments. Such a program can be issued via the Internet or networks. The program can be recorded on a computer-readable non-transitory recording medium such as a hard disk, floppy disk (FD) (trademark), CD-ROM, MO, DVD, and the like, and read from the recording medium, thereby capable of being executed by a computer. As a result, the same operation as that of the magnetic resonance imaging apparatus of the embodiment described above can be implemented.

According to at least one embodiment described above, image quality can be improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A magnetic resonance imaging apparatus comprising:
processing circuitry configured to
generate a pulse sequence including a plurality of repetition times (TRs) each of which includes an echo train and a driven equilibrium pulse applied following the echo train,
vary a flip angle of the driven equilibrium pulse,
obtain magnetic resonance image data collected by executing the pulse sequence, and
reconstruct a magnetic resonance image by using the magnetic resonance image data.

2. The magnetic resonance imaging apparatus according to claim 1, wherein the processing circuitry is configured to add a single IR (Inversion Recovery) pulse at a beginning of the entire pulse sequence including the plurality of the repetition times.

3. The magnetic resonance imaging apparatus according to claim 1, wherein the processing circuitry is configured to execute a T1-weighted sequence and vary a flip angle of the driven equilibrium pulse included in the T1-weighted sequence.

4. The magnetic resonance imaging apparatus according to claim 1, wherein the processing circuitry is configured to simulate an excitation signal in each repetition time to obtain a simulation signal, and calibrate, by using the simulation signal, the magnetic resonance image data.

5. The magnetic resonance imaging apparatus according to claim 4, wherein the processing circuitry is configured to simulate the excitation signal by Bloch equation or extended phase graph method.

6. The magnetic resonance imaging apparatus according to claim 1, wherein the processing circuitry is configured to extract one echo from the echo train in each of the repetition times as a template echo and calibrate the obtained magnetic resonance image data by using the template echo.

7. The magnetic resonance imaging apparatus according to claim 1, wherein the processing circuitry is configured to decrease flip angles of driven equilibrium pulses in the plurality of repetition times included in the pulse sequence with a lapse of time.

8. The magnetic resonance imaging apparatus according to claim 7, wherein the processing circuitry is configured to exponentially decrease the flip angles.

9. A magnetic resonance imaging method comprising:
executing a pulse sequence including repetition times (TRs) each of which includes an echo train and a driven equilibrium pulse applied following the echo train, while varying a flip angle of the driven equilibrium pulse;
obtaining magnetic resonance image data collected by executing the pulse sequence; and
reconstructing a magnetic resonance image by using the magnetic resonance image data.
